(19) United States
(12) Patent Application Publication
(10) Pub. No.: US 2024/0016518 A1
(43) Pub. Date: Jan. 18, 2024

(54) MEDICAL DEVICE INSUFFLATION ADAPTOR

(71) Applicant: Intuitive Surgical Operations, Inc., Sunnyvale, CA (US)

(72) Inventors: Douglas S. Langley, Millford, CT (US); Heath Corbet, Campbell, CA (US); Humphrey W. Chow, Cupertino, CA (US)

(21) Appl. No.: 18/038,902

(22) PCT Filed: Nov. 30, 2021
(86) PCT No.: PCT/US2021/061162

§ 371 Date: May 25, 2023

Related U.S. Application Data
(60) Provisional application No. 63/119,431, filed on Nov. 30, 2020.

(51) Int. Cl. A61B 17/34; A61M 39/10; A61B 17/00

(52) U.S. Cl. CPC A61B 17/3474; A61M 39/10; A61M 2039/1077; A61B 2017/00477

(57) ABSTRACT

A medical device includes an elbow, a first low-flow medical fluid fitting, and a first high-flow medical fluid fitting. The elbow includes a first end, a second end opposite the first end, and a bend between the first end and the second end. The first low-flow medical fluid fitting is coupled to the first end of the elbow and configured to be coupled to and seal with a complimentary second low-flow medical fluid fitting at a proximal end of a pneumocavity needle. And, the first high-flow medical fluid fitting is coupled to the second end of the elbow and configured to be coupled to and seal with a complimentary second high-flow medical fluid fitting of a high-flow medical fluid line.

### CLAIM OF PRIORITY

This application claims the benefit of priority to U.S. Patent Application Ser. No. 63/119,431, filed on Nov. 30, 2020, which is incorporated by reference herein in its entirety.

### TECHNICAL FIELD

This document relates generally to medical devices, and more particularly, to devices and methods related to establishing pneumoperitoneum in minimally-invasive laparoscopic surgical procedures.

### BACKGROUND

A surgical procedure, such as a minimally-invasive laparoscopic surgical procedure, may involve insufflation of a portion of the body with a gas. For example, in a laparoscopic procedure, an insufflation gas may be delivered to the peritoneal cavity of a patient to distend the abdomen, which may improve visual and physical access to internal organs in the abdomen. For example, distension of the patient's abdomen may provide sufficient operating space to enable adequate visualization of the structures and manipulation of instruments inside a patient.

Minimally-invasive laparoscopic surgical procedures may employ surgical systems that operate at least in part with computer-assisted control (“telesurgical systems” or “teleoperated surgical systems”). Such systems are sometimes referred to as robotic surgical systems or surgical robots. The da Vinci® Surgical Systems commercialized by Intuitive Surgical, Inc. are examples of such telesurgical systems.

Various telesurgical system architectures exist, including architectures that enable multiple surgical instruments to enter the body through a single body opening, sometimes referred to as “single-port” systems (e.g., the da Vinci SP® Surgical System), and architectures that enable multiple surgical instruments to enter the body individually at corresponding multiple locations, sometimes referred to as “multi-port” systems (e.g., the da Vinci Xi® Surgical System). In both so-called “single-port” and “multi-port” systems, surgical instruments commonly access a body cavity of a patient via one or more cannulas. Single-port systems typically mount a cannula to a wound retractor disposed in an incision in the body, while multi-port systems usually have cannulas directly inserted through the bodywall/incision. The cannula may also receive/be coupled to a surgical instrument access/seal device, which is configured to seal the opening in the body of the patient and to allow sealed entry/access to the body cavity by surgical instruments. Such instrument access/seal devices may include insufflation fittings for coupling the instrument access/seal device to a source of insufflation gas, which is employed to keep the cavity inflated throughout the surgical procedure. Prior to employing such teleoperated surgical instruments in a procedure initiated via the abdomen, the surgeon or other clinician needs to establish pneumoperitoneum in the body cavity of the patient. Pneumoperitoneum is pneumatosis (the abnormal presence of air or other gas) in the peritoneal cavity. Establishing pneumoperitoneum may involve a number of different approaches to laparoscopic access, including the Veress needle technique (also referred to as “Closed Entry”). The Veress needle technique generally includes inserting a Veress needle (sometimes referred to as Veres needle) into the peritoneal cavity and coupling the proximal (outside the body) end of the needle to an insufflation gas line, which supplies insufflation gas to inflate the body cavity.

### SUMMARY

When establishing pneumoperitoneum in a patient using a Veress needle, the needle needs to be carefully held stationary after being inserted through a manually retracted portion of the abdomen while the insufflation line is arranged adjacent and connected to the needle. The Veress needle-to-insufflation line connection commonly employs industry standard Luer lock fittings/connectors. At least some such Luer-Standard fittings/connectors are governed by ISO 80369-7-2016, ISO Luer Standard (Small-bore connectors for liquids and gases in healthcare applications—Part 7: Connectors for intravascular or hypodermic applications).

Even after attaching the insufflation line to the needle the assembly commonly needs to be held fixed while the gas is slowly pumped into the body cavity. This task may require a relatively high amount of dexterity and carries risks if there is slippage or fumbling of the parts.

For example, the insufflation tubing of an insufflation line can be stiff and have shape memory making it especially difficult to maneuver and attach to the Veress needle with a single hand (the typical use-case in relevant procedures). In addition, current insufflation line designs commonly orient the tubing of the insufflation line axially aligned with the vertically oriented Veress needle. Since the Veress needle is usually inserted perpendicular to the patient's abdomen, on top of difficult maneuvering, attaching the insufflation line can create a large arc or bend in the insufflation tube extending from the proximal end of the Veress needle. Such an arc/bend in the insufflation tubing can be both difficult to manage and have a higher likelihood of breaching the upper sterile boundary of a user (e.g., face, upper arms).

With an axial/inline connection between the Veress needle and insufflation line and only one free hand to use, the user may need to either manipulate the relatively heavy and stiff tubing by gripping the needle-to-insufflation line coupling tightly with the index and thumb while trying to turn it over the stationary needle or else grip the tubing vertically downwards (i.e., pronated wrist) while using the index and thumb to turn the coupling to attach the insufflation line to the needle.

Many of the complications associated with operative laparoscopy arise from creation of the pneumoperitoneum, such as subcutaneous emphysema and gas embolism, or from injury to internal structures during abdominal entry. In view of the relative risk of complications from establishing pneumoperitoneum, devices and methods that improve this process can have a substantial beneficial impact on risks associated with laparoscopic surgical procedures.

Examples according to this disclosure include insufflation line adaptors configured to be coupled between a Veress needle and an insufflation line and to orient the insufflation tubing of the insufflation line at approximately a 90-degree angle relative to the vertically oriented needle. By rotating the connection point of the tubing to the Veress needle 90 degrees, the potential arc/bend in the insufflation tube may be greatly reduced or even removed. In addition, a more ergonomic position may be achieved for holding the tubing while connecting the insufflation line to the Veress needle. For example, the hand of the user can be in a prone position gripping the tubing horizontally with the middle, ring, and pinky fingers while using the index and thumb to manipulate the coupling between needle and insufflation line. This natural gripping position offers greater control of both the sterile tubing and the connection onto the needle, which may function to reduce the likelihood of or prevent accidental slippage into non-sterile areas and improve the stability of the Veress needle as the insufflation line is connected thereto via example adaptors in accordance with this disclosure.

The beneficial impact of this rotated connection between Veress needle and insufflation line can increase with the use of larger diameter insufflation tubing in high-flow systems or tubing that contains advanced features such as humidification or heating elements. Thus, in examples, insufflation line adaptors in accordance with this disclosure can be configured to connect to and be packaged (e.g., in a kit) with high-flow insufflation tubing, which may be employed after establishing pneumoperitoneum to connect the insufflation line to, for example, an instrument access/seal device used to receive and seal with teleoperated surgical instruments.

A medical device in accordance with this disclosure includes an elbow, a first low-flow medical fluid fitting, and a first high-flow medical fluid fitting. The elbow includes a first end, a second end opposite the first end, and a bend between the first end and the second end. The first low-flow medical fluid fitting is coupled to the first end of the elbow and configured to be coupled to and seal with a complimentary second low-flow medical fluid fitting at a proximal end of a pneumocavity needle. And, the first high-flow medical fluid fitting is coupled to the second end of the elbow and configured to be coupled to and seal with a complimentary second high-flow medical fluid fitting of a high-flow medical fluid line.

A medical device in accordance with this disclosure includes a first low-flow Luer-standard fitting, an elbow, and a first high-flow Luer-standard fitting. The first low-flow Luer-standard fitting is configured to be coupled to and seal with a complimentary low-flow Luer-standard fitting at a proximal end of a needle configured to convey insufflation fluid therethrough. The elbow is coupled to the first low-flow Luer-standard fitting and includes an elbow angle that is at least approximately 90 degrees relative to a central axis of the first low-flow Luer-standard fitting. And, the first high-flow Luer-standard fitting is coupled to the elbow and configured to be coupled to and seal with a complimentary high-flow Luer-standard fitting of a high-flow insufflation line.

A method in accordance with this disclosure includes connecting a high-flow medical fluid line to an adaptor. The adaptor includes an elbow, a first low-flow medical fluid fitting, and a first high-flow medical fluid fitting. The elbow includes a first end, a second end opposite the first end, and a bend between the first end and the second end. The first low-flow medical fluid fitting is coupled to the first end of the elbow. The first high-flow medical fluid fitting is coupled to the second end of the elbow and coupled to and sealed with a complimentary second high-flow medical fluid fitting of the high-flow medical fluid line. The method also includes connecting the first low-flow medical fluid fitting of the adaptor to a proximal end of a pneumocavity needle.

Each of these non-limiting examples can stand on its own or can be combined in various permutations or combinations with one or more of the other examples.

This Summary is intended to provide an overview of subject matter of the present patent application. It is not intended to provide an exclusive or exhaustive explanation of the invention. The detailed description is included to provide further information about various aspects of the inventive subject matter of the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A depicts a surgeon accessing the abdominal cavity of a patient via the Veress needle technique.

FIG. 1B is an exploded view schematically depicting a system including an example insufflation line adaptor in accordance with this disclosure.

FIG. 1C is a perspective view of the example insufflation line adaptor of FIG. 1B.

FIG. 1D is a section view of the insufflation line adaptor of FIGS. 1B and 1C connected to a Veress needle and high-flow insufflation line.

FIGS. 1E-IG schematically depict additional example insufflation line adaptors in accordance with this disclosure.

FIG. 2A is a perspective view depicting an example surgical instrument access/seal assembly.

FIG. 2B is a section view depicting the surgical instrument access/seal device of FIG. 2A with an insufflation line adaptor and high-flow insufflation line in accordance with this disclosure.

FIG. 3A is a plan view illustration of an example medical system in a surgical environment.

FIG. 3B is an illustration of an example manipulating system.

FIG. 3C is an illustration of an example user control system.

FIG. 3D is an illustration of an example auxiliary system.

FIG. 3E is an illustration of an example instrument.

### DETAILED DESCRIPTION

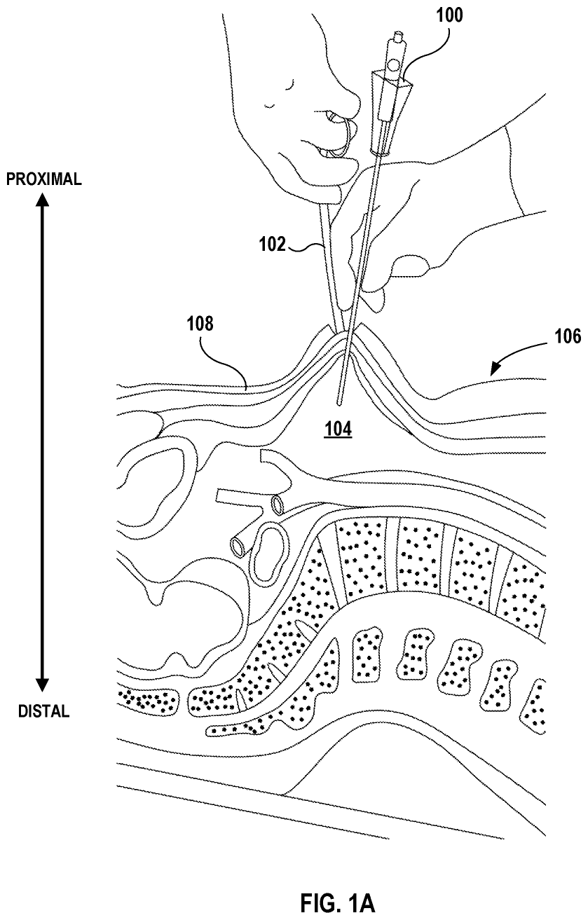

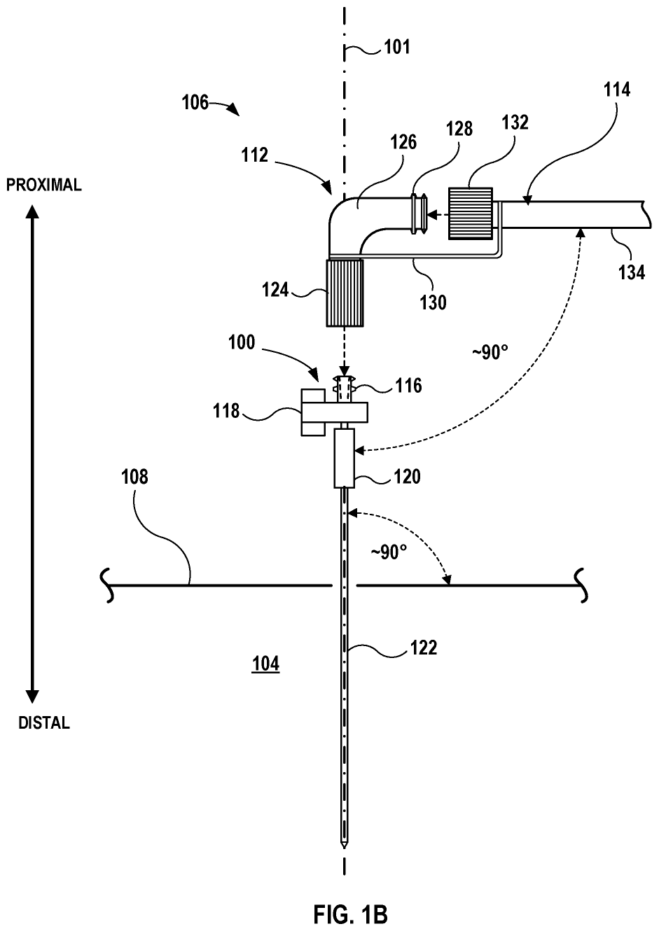

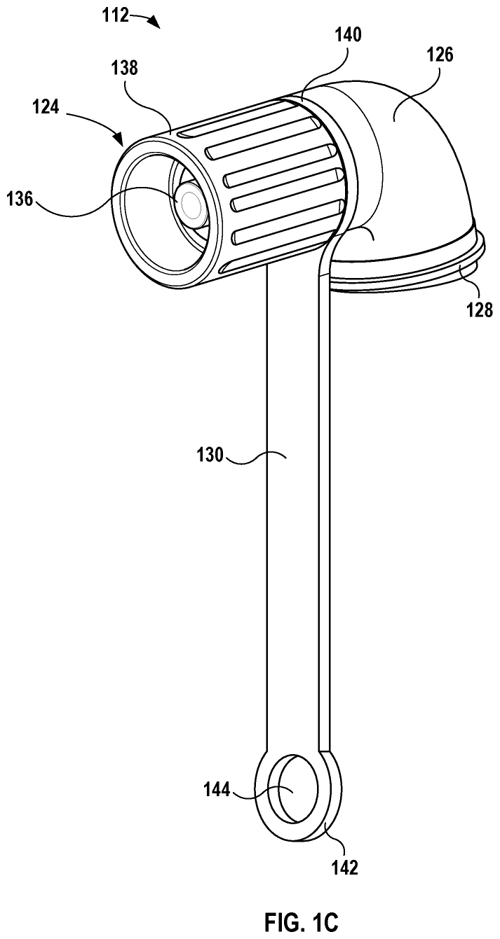

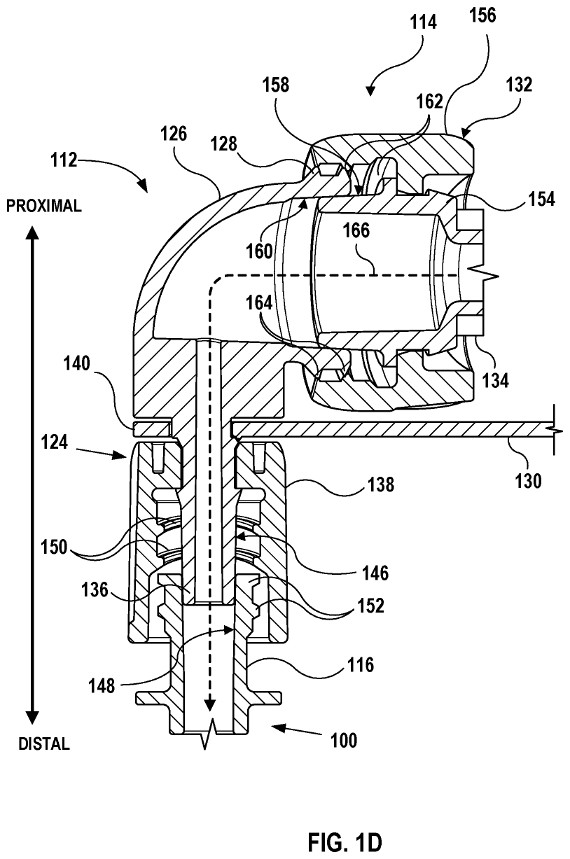

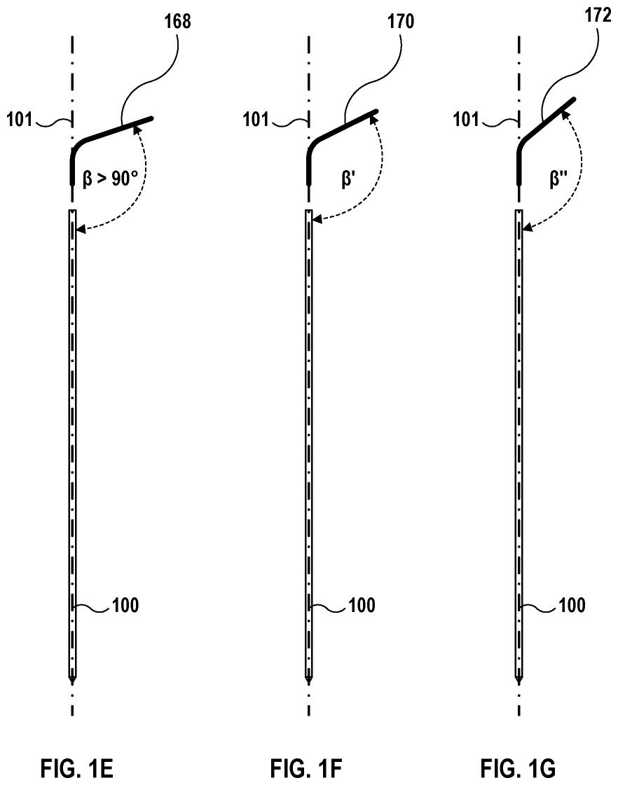

FIG. 1A depicts a surgeon accessing the abdominal cavity of a patient via the Veress needle technique. In FIG. 1A, Veress needle 100 and forceps 102 are employed to access the abdominal (peritoneal) cavity 104 of patient 106 through body wall 108. Forceps 102 may be employed to grasp and fix the skin of body wall 108 advantageously for a superficial incision and insertion of Veress needle 100.

In an example, to commence the procedure, a small incision (e.g., 3-5 millimeters—mm) is made in the umbilicus. In a patient with a Body Mass Index (BMI) in a normal range, to avoid the bifurcation of the inferior vena cava and/or aorta (or, more generally, the great vessels), Veress needle 100 is inserted at an angle of 45 degrees relative to horizontal. In a patient with an elevated BMI, Veress needle 100 is inserted at a 90-degree angle, since the umbilicus is caudal (i.e. further toward the tail or the posterior part of the body) to the bifurcation. As Veress needle 100 is inserted, the surgeon may feel/hear two “pops” at the rectus fascia and peritoneum, which is the serous membrane lining the peritoneal cavity. Depending upon circumstances of a particular patient and/or procedure, the surgeon might feel/hear two “pops” or may feel/hear just one or possibly even 3 “pops.” After traversing the peritoneum, the distal tip of Veress needle 100 should be within the peritoneal cavity.

Depending upon the angle of entry of Veress needle 100, Veress needle 100 is maintained at or reoriented to an angle of approximately 90 degrees to horizontal, which generally corresponds to the prone position of the body of the patient during the procedure. In some cases, a syringe filled with saline will be positioned at the proximal end of Veress needle 100 and employed to hydrate and aspirate the passage through the needle into peritoneal cavity 104. As described in detail with reference to FIG. 1B, an insufflation line is connected to the proximal end of Veress needle 100 and insufflation gas (e.g. CO₂) is pumped through the insufflation line and Veress needle 100 into peritoneal cavity 104 at a relatively low initial pressure (e.g., 10 millimeters of mercury—mmHg), after which the insufflation pressure can be raised (e.g., to 15 mmHg) to inflate the cavity.

FIG. 1B is an exploded view schematically depicting system 110 including Veress needle 100, example insufflation line adaptor 112 in accordance with this disclosure, and high-flow insufflation line 114 (more generally, high-flow medical fluid line). In FIG. 1B, Veress needle 100 includes low-flow female Luer fitting 116, stop cock valve 118, spring-loaded mechanism 120, and needle 122. Insufflation line adaptor 112 includes low-flow Luer lock connector 124, elbow 126, high-flow female Luer fitting 128, and tether 130. High-flow insufflation line 114 includes high-flow Luer lock connector 132 and insufflation tube 134.

Veress needle 100 is an example of a “pneumocavity” needle. In this disclosure, a “pneumocavity” needle is a needle used to introduce pneumotosis into a body cavity to create space. Although examples described in this disclosure reference the Veress Needle Technique and use of a Veress needle, examples in accordance with this disclosure can also be employed with other “pneumocavity” needles and employed in other types of procedures.

In this disclosure, high-flow is a rate and/or volume of flow that is higher relative to low-flow. In the case of insufflation line adaptor 112 and other example adaptors in accordance with this disclosure, for a constant fluid pressure, high-flow female Luer fitting 128 and high-flow Luer lock connector 132 provide or enable a higher rate and/or volume of flow of fluid than low-flow female Luer fitting 116 and low-flow Luer lock connector 124.

As depicted in FIG. 1B and described and illustrated in further detail below, insufflation line adaptor 112 is configured to be coupled to and seal an insufflation passage between Veress needle 100 and insufflation line 114. For example, low-flow Luer lock connector 124 is configured to be removably coupled to low-flow female Luer fitting 116 at the proximal end of Veress needle 100. High-flow Luer lock connector 132 is configured to be removably coupled to high-flow female Luer fitting 128 of insufflation line adaptor 112. In this manner, insufflation line adaptor 112 connected to vertically oriented Veress needle 100 positions insufflation tubing 116 to extend from the adaptor and needle horizontally, or, in other words, at an angle of approximately 90 degrees relative to longitudinal axis 101 of Veress needle 100. Similarly, elbow 126 of insufflation line adaptor 112 includes an elbow angle (or bend with an angle) approximately equal to 90 degrees relative to a central axis of low-flow Luer lock connector 124 (which, when coupled to Veress needle 100, is the same as axis 101 of needle 100).

FIG. 1C is a perspective view of example insufflation line adaptor 112. In FIG. 1C, insufflation line adaptor 112 includes low-flow Luer lock connector 124, elbow 126, high-flow female Luer fitting 128, and tether 130. Low-flow Luer lock connector 124 includes low-flow male Luer fitting 136 and locking collar 138. Low-flow male Luer fitting 136 is connected to or integral with elbow 126. Locking collar 138 is rotatably connected to low-flow male Luer fitting 136 and/or elbow 126 and is concentrically aligned and surrounds low-flow male Luer fitting 136. Tether 130 includes proximal end 140 rotatably coupled to insufflation line adaptor 112. And, tether 130 includes distal end 142 having aperture 144 configured to be rotatably and slidingly connected to insufflation tube 134 of insufflation line 114 (see FIG. 1B).

FIG. 1D is a section view of insufflation line adaptor 112 connected to Veress needle 100 and high-flow insufflation line 114. In FIG. 1D, low-flow Luer lock connector 124 of insufflation line adaptor 112 is connected to low-flow female Luer fitting 116 at the proximal end of Veress needle 100. In this example, low-flow male Luer fitting 136 includes male taper seal surface 146 that engages and seals against female taper seal surface 148 of low-flow female Luer fitting 116 of Veress needle 100. Locking collar 138 includes inner (female) coupling portion 150, which via rotation of locking collar 138 engage outer (male) coupling portion 152 of low-flow female Luer fitting 116. Inner coupling portion 150 of locking collar 138 and outer coupling portion 152 of low-flow female Luer fitting 116 include threads. In one example, inner coupling portion 150 and outer coupling portion 152 include Luer-type threads. In another example, inner coupling portion 150 and outer coupling portion 152 include another type of thread, lugs, or another locking/coupling mechanism. Locking collar 138 secures low-flow male Luer fitting 136 in sealed engagement with low-flow female Luer fitting 116.

High-flow Luer lock connector 132 of high-flow insufflation line 114 is connected to high-flow female Luer fitting 128 of insufflation line adaptor 112. In this example, high-flow Luer lock connector 132 includes high-flow male Luer fitting 154 and locking collar 156. Locking collar 156 is rotatably coupled to high-flow male Luer fitting 154, which is coupled to insufflation tube 134. High-flow male Luer fitting 154 includes male taper seal surface 158 that engages and seals against female taper seal surface 160 of high-flow female Luer fitting 128 of insufflation line adaptor 112. Locking collar 156 includes inner (female) coupling portion 162, which via rotation of locking collar 156 engage outer (male) coupling 164 of high-flow female Luer fitting 128. Inner coupling portion 162 of locking collar 156 and outer coupling portion 164 of high-flow female Luer fitting 128 include threads. In one example, inner coupling portion 162 and outer coupling portion 164 include Luer-type threads. In another example, inner coupling portion 162 and outer coupling portion 164 include another type of thread, lugs, or another locking/coupling mechanism. Locking collar 156 secures high-flow male Luer fitting 154 in sealed engagement with high-flow female Luer fitting 128.

Insufflation line adaptor 112 connected to Veress needle 100 and high-flow insufflation line 114 defines a single insufflation channel 166 to convey insufflation gas into a body cavity of a patient, e.g. peritoneal cavity 104 of patient 106. The insufflation flow rate/volume capacity through insufflation channel 166 is defined, at least in part, by the minimum inner diameter of channel 166. When insufflation line adaptor 112 is connected to Veress needle 100, the minimum inner diameter of channel 166 is dictated by the inner diameter of low-flow male Luer fitting 136 of insufflation line adaptor 112 or the minimum inner diameter of Veress needle 100, whichever is smaller. While the distinction between and capability to employ high-flow and low-flow insufflation is not particularly relevant to or beneficial for establishing pneumoperitoneum via the Veress needle technique, the capability to employ high-flow insufflation may have benefits and advantages for the laparoscopic procedure that follows.

As discussed in more detail below, example insufflation line adaptors in accordance with this disclosure (including example adaptor 112) include a high-flow female Luer fitting, which configures such adaptors to be packaged and employed with a high-flow insufflation line like example insufflation line 114. Example adaptors can thereby be employed advantageously in establishing pneumoperitoneum and can be decoupled from the associated high-flow insufflation line. The high-flow insufflation line, tethered to the disconnected adaptor can be connected to a high-flow Luer fitting of, for example, an instrument access/seal device employed in the laparoscopic procedure that follows establishing pneumoperitoneum.

Although example insufflation line adaptors, Veress needle 100 and high-flow insufflation lines are described as including particular types and varieties of fluid fittings, e.g. male versus female, Luer-standard versus some other standard or proprietary medical fluid fitting, etc., more generally, examples according to this disclosure may vary the particular fittings employed in the examples of FIGS. 1B-1D. For example, low-flow male Luer fitting 136 of example insufflation line adaptor 112 could be a low-flow female Luer or other type of medical fluid fitting and low-flow female Luer fitting 116 of Veress needle 100 could be a low-flow male Luer or other type of medical fluid fitting of a Veress needle or another type of pneumocavity needle. Similarly, high-flow female Luer fitting 128 of insufflation line adaptor 112 could be a high-flow male Luer or other type of medical fluid fitting and high-flow male Luer fitting 154 of high-flow Luer lock connector 132 of high-flow insufflation line 114 could be a high-flow female Luer or other type of medical fluid fitting of a high-flow fluid fitting connector of a medical fluid line.

As an example, a medical fluid line adaptor in accordance with this disclosure can include an elbow, a first low-flow medical fluid fitting, and a first high-flow medical fluid fitting. The elbow can include a first end, a second end opposite the first end, and a bend between the first end and the second end. The first low-flow medical fluid fitting is coupled to the first end of the elbow and configured to be coupled to and seal with a complimentary second low-flow medical fluid fitting at a proximal end of a pneumocavity needle. And, the first high-flow medical fluid fitting is coupled to the second end of the elbow and configured to be coupled to and seal with a complimentary second high-flow medical fluid fitting of a high-flow medical fluid line.

FIGS. 1E-1G schematically depict additional example insufflation line adaptors in accordance with this disclosure. The foregoing examples include an insufllation line adaptor that is configured to position the insufflation line connected thereto at approximately 90 degrees relative to the long axis of the Veress needle to which the adaptor is also connected. However, other example insufflation line adaptors in accordance with this disclosure can be configured to position the insufflation line at angles other than 90 degrees.

For example, example insufflation line adaptors, including example adaptors 168, 170, and 172 of FIGS. 1E-1F can be configured to position the insufflation line at angles, β, β′, β″ at least approximately 90 degrees relative to the long axis of Veress needle 100 to which each adaptor is also connected (or greater than approximately 90 degrees relative to a central axis of a low-flow male Luer fitting like low-flow male Luer fitting 136 of example adaptor 112). The advantages set forth above in reference to example adaptors including an approximate 90-degree elbow may also be achieved for adaptor angles in a range from and including approximately 90 degrees up to a threshold angle that is greater than 90 degrees and less than 180 degrees. Canting the adaptor elbow angle upward to greater than 90 degrees may improve the procedural and ergonomic advantages set forth above. For example, at an angle greater than 90 degrees, the user may more easily/comfortably/stably be able to manipulate the Luer lock connection between adaptor and Veress needle while also gripping the insufflation line extending from the adaptor.

In an example, an insufllation line adaptor in accordance with this disclosure includes an elbow angle that is greater than or equal to approximately 90 degrees and less than or equal to approximately 135 degrees. In another example, an insufflation line adaptor in accordance with this disclosure includes an elbow angle that is greater than or equal to approximately 90 degrees and less than or equal to approximately 120 degrees.

As noted above, it is important to maintain insufflation gas flow and pressure during laparoscopic procedures conducted after establishing pneumoperitoneum. In some cases, however, it may also be important to not exceed a threshold flow parameter, such as flow rates, flow volumes, insufflation pressures, etc. As an example, it may be important to not exceed an insufflation pressure of 80 mmHg and a steady state insufflation pressure of 15 mmHg. It is still possible, however, to vary the flow rate and/or volume, and it may be desirable to increase the flow rate and/or volume.

For example, a higher fluid flow rate/volume may be desirable or required in the event there is a leak in the system, or if there is another source of gas escape through the system. Higher flow may allow the system to maintain sufficient insufflation. An example of a particular application may be in trans-anal procedures in the colon. Insufflation applications that involve body cavities with greater sized holes/leaks may be improved with higher flow, because the higher flow can provide improved compensation for larger and/or a greater number of leaks in the cavity.

One Luer-standard fitting that can be used in insufflation applications is the so-called Luer taper. The Luer taper is a standardized system for certain fluid fittings used for making connections between a male-taper fitting and its mating female counterpart on medical and laboratory instruments. With standard Luer-type fittings, there's a limit to fluid flow through the fitting, which is dictated at least in part by the minimum diameter of the flow channel formed when the fittings are coupled together. In the Luer-type fitting this minimum diameter is typically the inner diameter of the tapered male Luer-type fitting, as described above in reference to FIG. 1D.

Since Luer-type fittings are a standard in the medical device industry, current medical devices are typically designed to fit such standard fittings, such as with low-flow female Luer fitting 116 of Veress needle 100 and the associated low-flow Luer lock connector 124 having low-flow male Luer fitting 136. This common design allows devices from different manufacturers (e.g., sources of insufflation gas, vacuum, or irrigation liquid, etc.; cannula seal connections, suction/irrigation instruments, etc.) to be coupled together. But since such standard fittings have fluid flow limits, new fittings may advantageously be configured to adapt/couple to both a higher flow fitting and to a standardized low-flow Luer-type fitting for retrofit capability.

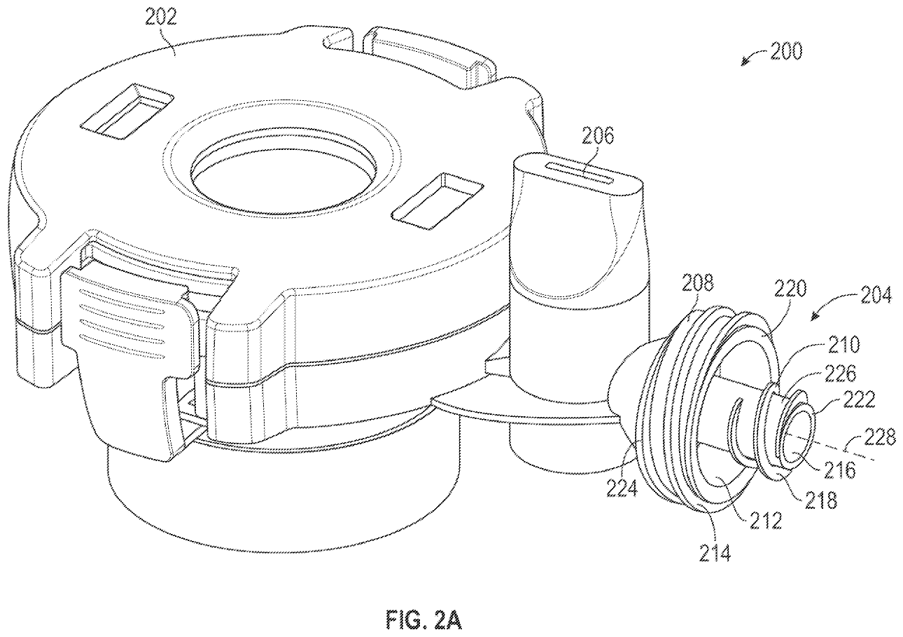

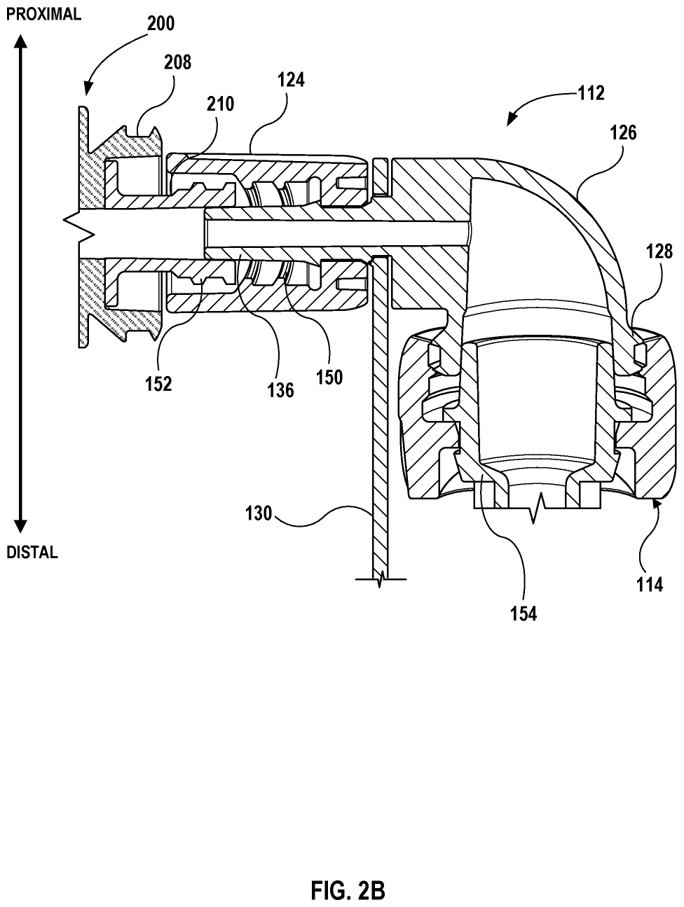

FIG. 2A is a perspective view depicting example surgical instrument access/seal device 200. In FIG. 2A, instrument seal device 200 includes device body 202, insufflation fitting 204, and valve 206. Valve 206 is fluidically connected to the fluid passageway of insufflation fitting 204 and configured to control the flow of insufflation gas into device body 202. Valve 206 can be a variety of different valves, including a stopcock valve.

Insufflation fitting 204 extends from device body 202, and a single flow passage is defined through fitting 204 and into device body 202. Insufflation fitting 204 includes high-flow fitting 208 and low-flow fitting 210. As noted, in this disclosure, high-flow is a rate and/or volume of flow that is higher relative to low-flow. In the case of insufflation fitting 204 and other such fittings in accordance with this disclosure, for a constant fluid pressure high-flow fitting 208 provides or enables a higher rate and/or volume of flow of fluid than low-flow fitting 210. In some examples, high-flow fitting 208 and low-flow fitting 210 are fabricated integral with one another to form insufflation fitting 204 in accordance with this disclosure. In another example, however, low-flow fitting 210 may be fabricated as a separate component and then be coupled to high-flow fitting 208. For example, low-flow fitting 210 may be fabricated as a separate component and then be coupled to high-flow fitting 208 using, for example, an adhesive or by welding low-flow fitting 210 to high-flow fitting 208.

In the example of insufflation fitting 204, high-flow fitting 208 includes first internal/inner (sometimes referred to as “female”) sealing surface 212 and first coupling portion 214. Low-flow fitting 210 includes second internal/inner sealing surface 216 and second coupling portion 218. First scaling surface 212 of high-flow fitting 208 is a female tapered surface, the diameter of which decreases from free end 220 of high-flow fitting 208 toward valve 206 and device body 202. Additionally, first sealing surface 212 is an annular sealing surface. Similarly, second sealing surface 216 of low-flow fitting 210 is a female tapered surface, the diameter of which decreases from free end 222 of low-flow fitting 210 toward high-flow fitting 208, valve 206, and device body 202. Second sealing surface 216 is also an annular sealing surface.

First coupling portion 214 is on first external surface 224 of high-flow fitting 208, which external surface is generally reverse of first internal sealing surface 212. Second coupling portion 218 is on second external surface 226 of low-flow fitting 210, which external surface is generally reverse to second internal sealing surface 216. First coupling portion 214 of high-flow fitting 208 and second coupling portion 218 of low-flow fitting 210 include threads. In one example, first coupling portion 214 and second coupling portion 218 include Luer-type threads. In another example, first coupling portion 214 and second coupling portion 218 include another type of thread, lugs, or another locking/coupling mechanism.

Low-flow fitting 210 is concentric with and nested partially within high-flow fitting 208. High-flow fitting 208 and low-flow fitting 210 share a common centerline, which also defines a single flow path centerline through the flow passage 230 of insufflation fitting 204.

FIG. 2B is a section view depicting surgical instrument access/seal device 200, insufflation line adaptor 112 in accordance with this disclosure, and high-flow insufflation line 114. In FIG. 2B, low-flow male Luer fitting 136 of insufflation line adaptor 112 is mated with low-flow female Luer fitting 210. While this connection is possible, the arrangement of components in FIG. 2B is not the optimal manner to connect high-flow insufflation line 114 to instrument access/seal device 200. To reduce confusion and time delays prior to or during complex laparoscopic procedures, locking collar 138 of low-flow Luer lock connector 124 of insufflation line adaptor 112 is configured to prevent adaptor 112 from being locked onto instrument access/seal device 200 in the manner depicted in FIG. 2B. In particular, the free end of locking collar 138 (the end closest to device 200) is configured to run into high-flow female Luer fitting 208 before inner coupling portion 150 (e.g., threads) of locking collar 138 can engage and lock onto second (outer) coupling portion 218 (e.g., threads) of low-flow female Luer fitting 210.

In the event insufflation line adaptor 112 and high-flow insufflation line 114 are connected to instrument access/seal device 200 in the manner depicted in FIG. 2B, the user is alerted to the error by not being able to lock low-flow male Luer fitting 136 to low-flow female Luer fitting 210 with locking collar 138 and can remove adaptor 112 from device 200, disconnect high-flow insufflation line 114 from adaptor 112, and correctly connect high-flow male Luer fitting 154 of high-flow insufflation line 114 to high-flow female fitting 208 of device 200.

In an example in accordance with this disclosure, a medical device (instrument access/seal device 200 is an example of such a medical device) includes an first low-flow medical fluid fitting having a radially outward surface generally reverse from a first inner sealing surface, the inner sealing surface defining a flow passageway for a medical fluid; and a first high-flow medical fluid fitting having a second inner sealing surface extending around and radially offset from the radially outward surface of the first low-flow medical fluid fitting.

A medical fluid line adaptor (insufflation line adaptors 112, 168, 170, and 172 are examples of such an adaptor) includes an elbow including a first end, a second end opposite the first end, and a bend between the first end and the second end; a second low-flow medical fluid fitting coupled to the first end of the elbow and configured to be coupled to and seal with a complimentary third low-flow medical fluid fitting at a proximal end of a pneumocavity needle; a locking collar rotatably coupled to the second low-flow medical fluid fitting; and a second high-flow medical fluid fitting coupled to the second end of the elbow and configured to be coupled to and seal with a complimentary third high-flow medical fluid fitting of a high-flow medical fluid line.

On condition that the second low-flow medical fluid fitting of the medical fluid line adaptor is positioned to engage and/or be coupled to the first low-flow medical fluid fitting of the medical device, the locking collar engages the first high-flow medical fluid fitting of the medical device to prevent the second low-flow medical fluid fitting of the medical fluid line adaptor from being coupled to and sealed with the first low-flow medical fluid fitting of the medical device.

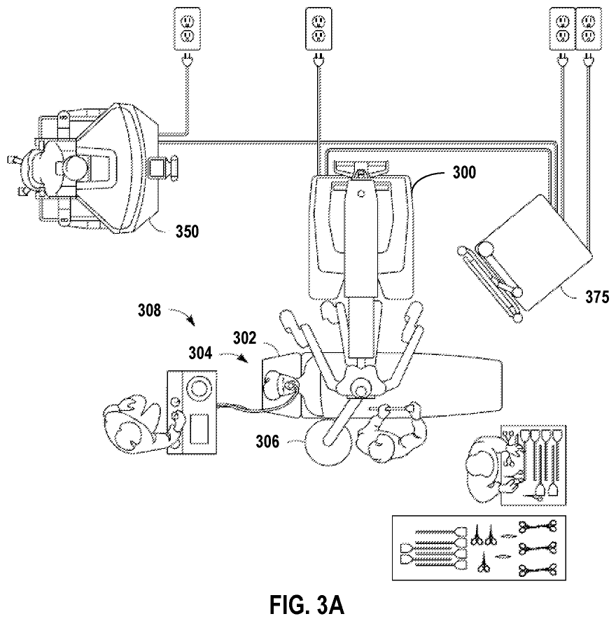

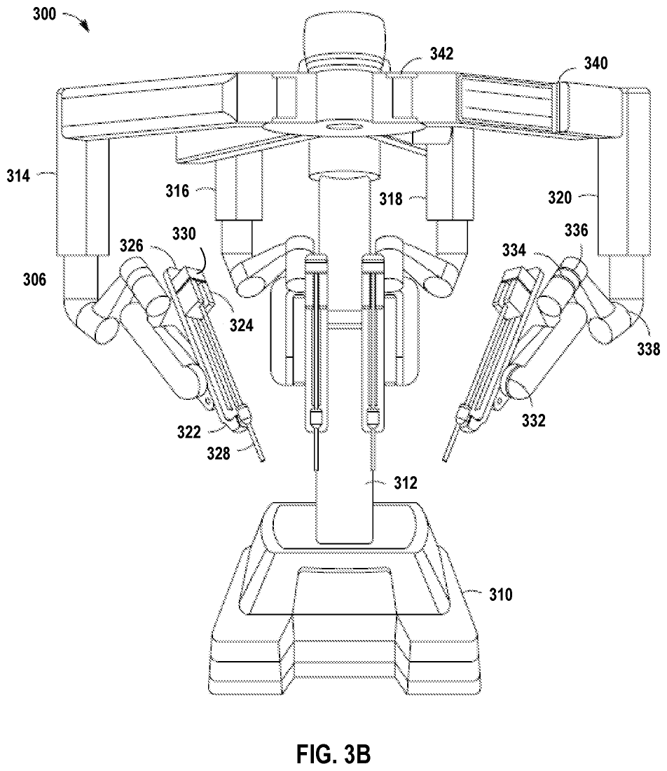

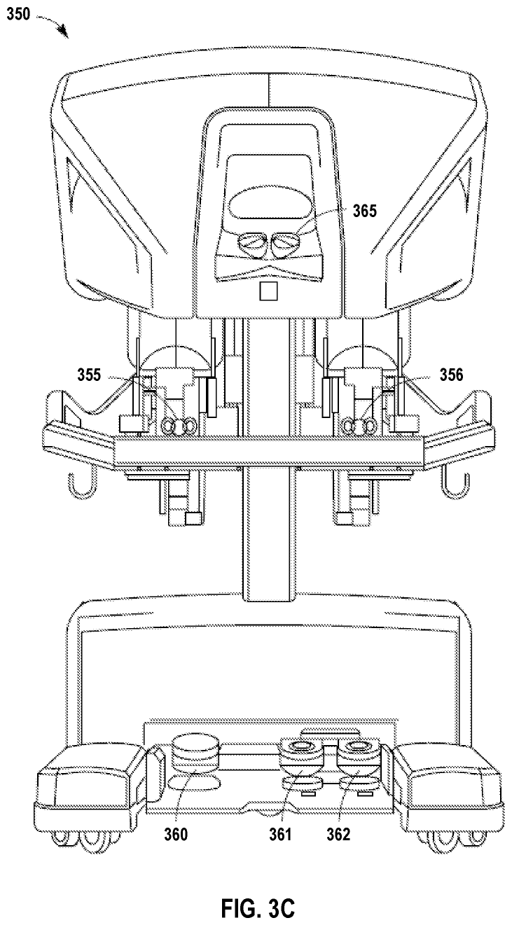

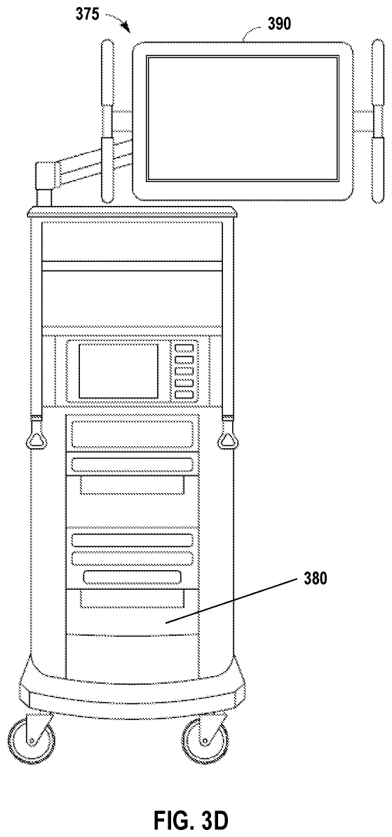

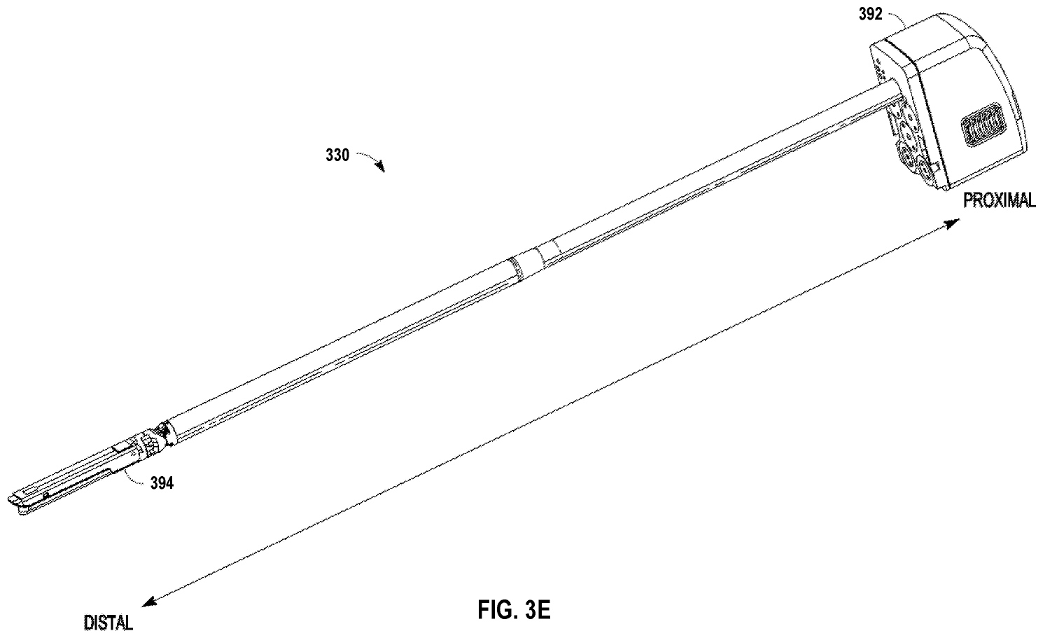

FIG. 3A is a plan view depicting an example medical procedure environment that includes a multi-arm manipulating system 300 adjacent to a surgical table 302 that supports a patient 304. The medical environment of FIG. 3A and systems/components of FIGS. 1B-1D may be employed after establishing pneumoperitoneum using a Veress needle and example insufflation line(s) and adaptor(s) in accordance with examples of this disclosure. Referring again to FIG. 3A, a second manipulating system 306 may also be situated at the surgical table 302. The manipulating systems 300, 306 may be free-standing on a movable base, or they may be mounted to a table, floor, wall, or ceiling, or they may be supported on another piece of equipment in the clinical environment.

The manipulating system 300 or system 306 may be part of a larger system 308, which may include other sub-systems, including, for example, fluoroscopy or other imaging equipment. One or both of the manipulating systems 300, 306 may be operatively coupled to a user control system 350 or an auxiliary system 375, or both. The user control system 350 may include one or more user input devices (e.g., controls) that may be configured to receive inputs from a user (e.g., clinician). The user control system 350 may also include or one or more user feedback devices (e.g., viewing system, or tactile or auditory feedback system) that may be configured to provide information to the user regarding the movement or position of an end effector, or an image of a surgical area. The auxiliary system 375 may, for example, include computer processing equipment (e.g., a processor circuit or graphics hardware), or communication equipment (e.g., wired or wireless communication circuits), or endoscopic camera control and image processing equipment.

FIG. 3B depicts example manipulating system 300. The example manipulating system 300 includes a base 310, a support tower 312, and one or more manipulator arms 314, 316, 318, 320, which may be mounted on the support tower 312. An instrument 330 (shown in more detail in FIG. 3E) is mounted to an instrument mount 322 on one of the manipulator arms 314, 316, 318, 320. The instrument mount 322 includes, as an example, an instrument carriage 324, which is mounted to a spar 326, which may be a telescoping or non-telescoping spar. A cannula 133 may be mounted to a cannula mount 126, and the instrument 330 may be inserted through a cannula seal in the cannula 328, and into the patient 304 (FIG. 3A) for use in a therapeutic or diagnostic surgical procedure. Through movement of the manipulator arms 314, 316, 318, 320, the translation and orientation of the instrument 330 may be controlled in multiple mechanical degrees of freedom, e.g. lateral, horizontal, vertical, angular movements in one, two, or three planes. The system 300 may include one or more light features 332, 334, 336, 338, 340, 342 at one or more of a variety of locations on the manipulator arms 314, 316, 318, 320 (i.e., at joints between arm links, as shown).

Cannula 328 may be inserted into the patient 304, and a surgical instrument access/seal device (not shown) is inserted into the cannula. The instrument seal prevents insufflation gas from escaping through the open cannula when no instrument is inserted in the cannula, and it also prevents insufflation gas from escaping between the instrument shaft and the cannula inner wall when an instrument is inserted in the cannula.

FIG. 3C depicts example user control system 350. The user control system 350 includes hand controls 355, 356 and foot pedal controls 360, 361, 362. The hand controls 355, 356 and foot pedal controls 360, 361, 362 are used to control equipment at one or more of the manipulating systems 300, 306. For example, an operator may manipulate portions of a distal end of an instrument 330 by using the instrument controls. The controls may include haptic feedback features so that a surgeon may interpret physical information at the instrument 330, such as resistance or vibration, through the controls. The user control system 350 may also include a viewing system 365 that displays video or other images of a surgical site.

FIG. 3D depicts an example auxiliary system 375. The example auxiliary system 375 optionally includes telesurgical system functions that are not incorporated into other system units, such as computer processing system 380 for processing teleoperation controls, facilitating communication between the user control system and the manipulating system, or a remote site, endoscopic camera control and illumination, electrosurgical generation and control, etc. The auxiliary system 375 may also include a display 390, which shows images that the user (e.g., a clinician) is seeing on the user control system 350, a video feed from a camera in the patient 304, or other information. In an example configuration, signals input at a user control system 350 may be transmitted to the processing system 380 on the auxiliary system 375, which interprets the inputs and generate commands that are transmitted to the manipulating system 300 to cause manipulation of an instrument 330 or portions of a manipulator arm 314. The processing system 380 is shown on a cart for exemplary purposes, but it may also be arranged in various configurations, e.g., it may be integrated as part of the user control system 350, the manipulating system 300, 306, or both, or divided between the user control system 350 and manipulating system 300, 306. The equipment may also be provided as software, hardware, or both, on an installed or remote system.

FIG. 3E depicts example instrument 330. The instrument 330 includes a proximal portion 392, which is configured to couple to an instrument mount on a manipulator arm. The instrument 330 also includes a distal portion 394 and an instrument shaft 196 between the proximal portion 392 and the distal portion 394. The distal portion 394 shown is a stapler, and in other instruments it may be a cautery tool, cutter, camera, or other medically relevant end effector. The instrument 330 may be teleoperatively controlled via command signals received from a control computer, such as a user control system 350 or auxiliary system 375 to conduct a surgical procedure. Inputs may be received from a user (e.g., clinician), and the instrument 330 may be controlled based on the user inputs.

In an example, instrument 330 is inserted into the patient 330 via cannula 328, which also contains a surgical instrument access/seal device as described above. Insufflation of a body cavity of patient 330 can be established employing the Veress needle technique and insufflation line adaptors in accordance with this disclosure and can be maintained by decoupling the high-flow insufflation line from the adaptor and connecting the insufflation line to a high-flow Luer fitting on an instrument access/seal device.

Persons of skill in the art will understand that any of the features described above may be combined with any of the other example features, as long as the features are not mutually exclusive. All possible combinations of features are contemplated, depending on clinical or other design requirements. In addition, if manipulating system units are combined into a single system (e.g., telesurgery system), each individual unit may have the same configuration of features, or, one patient-side unit may have one configuration of features and another patient-side unit may have a second, different configuration of features.

The examples (e.g., methods, systems, or devices) described herein may be applicable to surgical procedures, non-surgical medical procedures, diagnostic procedures, cosmetic procedures, and non-medical procedures or applications. The examples may also be applicable for training, or for obtaining information, such as imaging procedures. The examples may be applicable to handling of tissue that has been removed from human or animal anatomies and will not be returned to a human or animal, or for use with human or animal cadavers. The examples may be used for industrial applications, general robotic uses, manipulation of non-tissue work pieces, as part of an artificial intelligence system, or in a transportation system.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. But, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description. Coordinate systems or reference frames are provided for aiding explanation, and implantations may use other reference frames or coordinate systems other than those described herein.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features ofa particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

1. A medical device comprising:
an elbow including a first end, a second end opposite the first end, and a bend between the first end and the second end;
a first low-flow medical fluid fitting coupled to the first end of the elbow and configured to be coupled to and seal with a complimentary second low-flow medical fluid fitting at a proximal end of a pneumocavity needle; and
a first high-flow medical fluid fitting coupled to the second end of the elbow and configured to be coupled to and seal with a complimentary second high-flow medical fluid fitting of a high-flow medical fluid line.

2. The medical device of claim 1, wherein:
the first low-flow medical fluid fitting and the first complimentary low-flow medical fluid fitting are each Luer-standard fittings.

3. The medical device of claim 1, wherein:
the first high-flow medical fluid fitting and the first complimentary high-flow medical fluid fitting are each Luer-standard fittings.

4. The medical device of claim 1, wherein:
the first low-flow medical fluid fitting, the first complimentary low-flow medical fluid fitting, the first high-flow medical fluid fitting, and the first complimentary high-flow medical fluid fitting are each Luer-standard fittings.

5. The medical device of claim 1, wherein:
the first low-flow medical fluid fitting is a male fitting, and the first complimentary low-flow medical fluid fitting is a female fitting.

6. The medical device of claim 1, wherein:
the first high-flow medical fluid fitting is a female fitting, and the first complimentary high-flow medical fluid fitting is a male fitting.

7. The medical device of claim 1, wherein:
the pneumocavity needle is a Veress needle.

8. The medical device of claim 1, wherein:
the medical fluid line is an insufflation gas line.

9. The medical device of claim 1, wherein:
the bend of the elbow is at least 90 degrees relative to a central axis of the first low-flow medical fluid fitting.

10. The medical device of claim 1, wherein:
the medical device further comprises a first locking collar rotatably coupled to the first low-flow medical fluid fitting.

11. The medical device of claim 10, wherein:
the first locking collar includes a female coupling portion configured to engage and lock onto a male coupling portion of the first low-flow medical fluid fitting at the proximal end of the pneumocavity needle.

12. The medical device of claim 11, wherein:
the female coupling portion includes Luer-type threads.

13. The medical device of claim 1, wherein:
the medical device further comprises a tether including a first end and a second end opposite the first end;
the first end of the tether is coupled to the first low-flow medical fluid fitting; and
the second end of the tether is configured to be coupled to the high-flow medical fluid line.

14. The medical device of claim 13, wherein:
the first end of the tether is rotatably coupled to the first low-flow medical fluid fitting and the second end is configured to be rotatably and slidingly coupled to the high-flow medical fluid line.

15. The medical device of claim 10, wherein:
an end of the locking collar is configured to engage a portion of a high-flow Luer fitting to prevent the first low-flow medical fluid fitting from engaging and locking onto the high-flow Luer fitting.

16. The medical device of claim 1, wherein:
the first high-flow medical fluid fitting includes a first coupling portion configured to engage and lock onto a second coupling portion of a locking collar rotatably connected to the complimentary second high-flow medical fluid fitting of the high-flow medical fluid line.

17. The medical device of claim 1, wherein:
the first high-flow medical fluid fitting includes a third Luer taper seal surface configured to engage and seal with a fourth Luer taper seal surface of the complimentary second high-flow medical fluid fitting of the high-flow medical fluid line.

18. (canceled)

19. The medical device of claim 1, wherein:
the first high-flow medical fluid fitting includes a third Luer taper seal surface configured to engage and seal with a fourth Luer taper seal surface of the complimentary second high-flow medical fluid fitting of the high-flow medical fluid line.

20. (canceled)

21. A medical device comprising:
a first low-flow Luer-standard fitting configured to be coupled to and seal with a complimentary low-flow Luer-standard fitting at a proximal end of a needle configured to convey insufflation fluid therethrough;
an elbow coupled to the first low-flow Luer-standard fitting and including an elbow angle that is at least approximately 90 degrees relative to a central axis of the first low-flow Luer-standard fitting; and
a first high-flow Luer-standard fitting coupled to the elbow and configured to be coupled to and seal with a complimentary high-flow Luer-standard fitting of a high-flow insufflation line.

22. A method comprising:
connecting a high-flow medical fluid line to an adaptor, the adaptor including:
an elbow including a first end, a second end opposite the first end, and a bend between the first end and the second end;
a first low-flow medical fluid fitting coupled to the first end of the elbow; and
a first high-flow medical fluid fitting coupled to the second end of the elbow and coupled to and sealed with a complimentary second high-flow medical fluid fitting of the high-flow medical fluid line; and
connecting the first low-flow medical fluid fitting of the adaptor to a proximal end of a pneumocavity needle.
